(19) United States
(12) Patent Application Publication
(10) Pub. No.: US 2026/0102220 A1
(43) Pub. Date: Apr. 16, 2026

(54) MEDICAL TECHNOLOGY DEVICE WITH COLLISION SENSOR

(71) Applicant: Siemens Healthineers AG, Forchheim (DE)

(72) Inventors: Verena SCHMIDT, Erbendorf (DE); Claudia IGNEY, Erlangen (DE); Martin SEIFERT, Bayreuth (DE)

(73) Assignee: Siemens Healthineers AG, Forchheim (DE)

(21) Appl. No.: 19/356,414

(22) Filed: Oct. 13, 2025

(30) Foreign Application Priority Data
Oct. 14, 2024 (DE) 10 2024 209 958.9

(51) Int. Cl. A61B 90/00

(52) U.S. Cl. CPC A61B 90/06; A61B 2090/065

(57) ABSTRACT

A medical technology device has a cladding element, in which a sensor element is arranged. The sensor element is configured to detect when the cladding element collides with the base body or another body during movement of an additional part relative to the base body. The sensor element is configured to issue a sensor signal in the event of a collision of the cladding element. The cladding element has an inner shell and an outer shell, both of which include plastic, lie one on top of the other, and are joined together at their edge regions by way of a connection. The inner shell and the outer shell form a cavity therebetween, in which the sensor element is arranged to at least partially fill. The sensor element is configured to be removed from the cavity again after the connection between the inner shell and the outer shell is released.

### CROSS-REFERENCE TO RELATED APPLICATIONS

The present application claims priority under 35 U.S.C. §119 to German Patent Application No. 102024209 958.9, filed October 14, 2024, the entire contents of which is incorporated herein by reference.

### FIELD

One or more example embodiments of the present invention relate to a medical technology device,

wherein the device has a cladding element,

wherein a sensor element is arranged in the cladding element and serves to detect a collision of the cladding element during movement,

wherein the sensor element issues a sensor signal in the event of a collision of the cladding element,

wherein the cladding element has an inner shell and an outer shell, which both consist of plastic, lie one on top of the other, and are joined together in their edge regions by way of a connection.

### BACKGROUND

In the case of medical technology devices of the type cited in the introduction, the additional part (for example an extension arm) is displaced relative to the base body directly or by way of a number of intermediate elements. A collision may occur, for example with the base body or other bodies or even persons, during displacement of the additional part. If such a collision occurs, it is necessary to detect the collision and terminate the displacement movement as quickly as possible. The sensor element serves to detect such a collision. The sensor element turns the cladding element into what is known as a smart cladding element.

In the prior art, a sensor element is attached in a fixed (and often non-releasable) manner to an inner shell of the cladding element. The inner shell usually consists of a relatively hard material. A further layer is attached to the side of the sensor element facing away from the inner shell. In some cases, this further layer itself forms the outer shell. In other cases, the further shell is relatively soft and forms a type of crumple zone. In this case, the outer shell is attached in a fixed and non-releasable manner to the further layer.

Because the inner shell, the sensor element and the further layer are connected in a fixed and non-releasable manner, it is impossible, or at least only possible with great difficulty, to recycle the elements of which the cladding element is composed. Furthermore, various top coatings are usually required, which further impede recycling. For example, dismantling the cladding element may entail significant effort. It is also possible for materials to be incompatible, so that they have to be cut apart or dismantled before they can be recycled separately. In some cases, metal elements, for example elements made from copper, are also screwed into the cladding element. Such elements often have to be removed manually before recycling. In some cases, a copper coating is applied to the inner shell and/or the outer shell on the inside or outside. In this case, the shells concerned are contaminated with copper and cannot be recycled.

### SUMMARY

At least one object of one or more example embodiments of the present invention is to create possibilities by which such recycling is enabled or made easier.

Various aspects must be taken into consideration in order to achieve these objectives. One aspect is the selection of materials with a view to their recyclability: Materials should be selected so as to ensure a high level of recyclability. Recyclability refers to the ease with which a product can be dismantled into unmixed materials in order to facilitate the recycling process based thereon. The aim is to increase the recycling rate, for example because a high level of recyclability makes it easier to dismantle the individual parts of the cladding element into unmixed materials, thus creating the basis for a high recycling rate. The recycling rate specifies the portion of a specific product which can be recycled and/or the portion of a multiplicity of similar products which can be recycled. The recycling rate differs from the recycled content. The recycled content is the portion of a given product that consists of recycled material. A further aspect consists in enabling easy dismantling. Preferably, the design should therefore be modular in order to enable easy dismantling and separation of the materials and components. Furthermore, the modular design facilitates not only the recycling but also the repair and replacement of parts, for example a replacement of a sensor element or of a cover shell in the event of a defect. Under certain circumstances, individual modules/components of a cladding element can even be reused without further processing, for example without shredding the inner shell and/or the outer shell.

At least the above-mentioned object is achieved by a medical technology device having the features of the independent claims. Advantageous embodiments of the device form the subject matter of the dependent claims.

Independent of the grammatical term usage, individuals with male, female or other gender identities are included within the term.

According to one or more example embodiments of the present invention, a medical technology device of the type cited in the introduction is embodied such

that the inner shell and the outer shell form a cavity therebetween,

that the sensor element is arranged in the cavity so that it fills the cavity partially but not fully, and

that the sensor element, although fastened in the cavity, can be removed from the cavity again after the connection between the inner shell and the outer shell is released.

Preferably, so that it can easily be removed again from the cavity, the sensor element is not adhesively bonded to the inner shell or the outer shell. A water-soluble adhesive, for example, can be used if adhesive bonding is required.

The sensor element can be embodied in many different ways with regard to its shape and mode of operation. The decisive factor is the ability to detect via the sensor element when pressure is exerted onto the cladding element from outside. The sensor element can be embodied for example as a pressure sensor, as a large-surface switch or as a safety strip. Large-surface switches and safety strips are known per se. Other types of sensor elements are also possible, for example so-called tactile textile sensors and so-called smart textiles or ultra-thin 3D printed textiles.

Provision is made in particular for the inner shell, the outer shell and/or the sensor element to be able to be separated and/or divided in a non-destructive manner in order to release the connection.

Able to be separated in a non-destructive manner" is understood in particular to mean that the separated components have no substantial quality losses. As a result, they can be reused and/or recycled in full or in part.

The inner shell and the outer shell preferably consist of materials with a recycled content of at least 30%. This enables the environmental "footprint" to be reduced. The recycled content can also be greater than 30%, for example at least 40%, at least 50%, at least 60%, at least 70%, at least 80% and also at least 90%. The inner shell and the outer shell ideally consist entirely or almost entirely (97% and more) of such materials. The material can be made available for example in PIR (post-industrial recycled) and PCR (post-consumer recycled) form. The recycling processes can work mechanically, with pyrolysis or with depolymerization.

In many cases, the inner shell and the outer shell have an identical three-dimensional curved shape. In this case, the sensor element is preferably held and fastened in the cavity on account of the molding of the inner shell and the outer shell. This embodiment makes it easier to fasten the sensor element in the cavity.

The inner shell and/or the outer shell preferably have positioning elements which interact with corresponding counter-elements of the sensor element such that the sensor element is fastened on account of the interaction of the positioning elements and the counter-elements relative to the inner shell and/or the outer shell. As a result, an exact positioning of the sensor element can be ensured. The positioning elements are preferably arranged on the inner shell. The positioning elements can be for example nubs, the counter-elements recesses. The positioning elements and the counter-elements can however also be embodied inversely.

The sensor element preferably has intended break points, along which the sensor element breaks or tears when a laterally acting tensile force is exerted on the sensor element while the sensor element is disposed between the inner shell and the outer shell. As a result, the sensor element can easily be removed from the cavity either completely in several large parts or almost completely in one large part. This embodiment is relevant in particular in connection with the embodiment in which the positioning elements and the counter-elements are also present.

The connection between the inner shell and the outer shell can be embodied as required. It is usually an adhesively bonded connection or a latching connection. If a fluid-tight connection and where applicable even a gas-tight connection is required, the gas-tightness and fluid-tightness in the latter-cited case can be ensured by an additional seal element, for example a permanently elastic element extending circumferentially in a ring-shaped manner in the outer region.

The inner shell preferably consists of a material with flameproofing. The flameproofing preferably corresponds to at least UL94-V1 (as of August 2024), even better UL94-V0. The flameproofing can be achieved for example by supplementing a per se flammable base material of the inner shell with a non-flammable or low-flammability additional material. The outer shell, on the other hand, can in this situation consist of a material without flameproofing. On account of the circumstance that the outer shell can consist of a material without flameproofing, a very high recycled content of for example at least 90% can be achieved for the outer shell.

The movement of the additional part relative to the base body is typically effected by a control facility (also referred to as a control device) of the device through the corresponding actuation of a number of drives. In this case, the sensor signal is output to the control facility of the device. When the sensor signal is transmitted, the control facility terminates or inverts the movement of the additional part relative to the base body.

The inner shell and/or the outer shell and/or the sensor element are preferably recyclable. This is in particular also the case if the inner shell and/or the outer shell are reusable without shredding after dismantling of the cladding element.

The cladding element preferably has a modular structure. This makes it possible to replace individual components of the cladding element in particular in the event of a defect.

### BRIEF DESCRIPTION OF THE DRAWINGS

The above-described properties, features and advantages of this invention as well as the manner in which they are achieved will become clearer and more comprehensible in conjunction with the following description of the exemplary embodiments, which are explained in more detail in conjunction with the drawings. In the drawings, in schematic representation:

FIG. 1 shows a medical technology device,

FIG. 2 shows a section through an additional part along a line II-II in FIG. 1,

FIG. 3 shows a section through a cladding element,

FIG. 4 shows a flow diagram,

FIG. 5 shows a top view of an inner shell and a sensor element, and

FIG. 6 shows a section through a cladding element.

### DETAILED DESCRIPTION

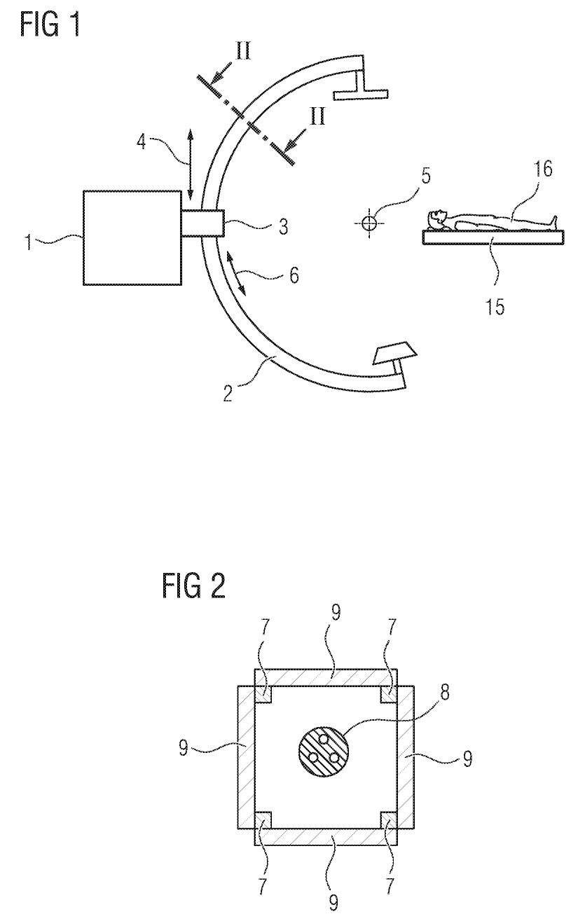

According to FIG. 1, a medical technology device has a base body 1 and at least one additional part 2. The additional part 2 is movable relative to the base body 1. In the example illustrated, the medical technology device is for example a C-arm system, the C-arm of which is the additional part 2. Here, the additional part 2 is connected to the base body 1 by way of an intermediate element 3. For example, the intermediate element 3 can be height-adjustable on the base body 1. This is indicated in FIG. 1 by a double arrow 4. The additional part 2 can likewise be pivoted about a pivot axis 5 in a guide of the intermediate element 3. This is indicated in FIG. 1 by a double arrow 6.

According to FIG. 2, the additional part 2 has an inner structure, for example support struts 7 and cables 8 guided on the inside of the additional part 2. The additional part 2 furthermore has (at least) one cladding element 9. The inner structure is cladded via the cladding element 9.

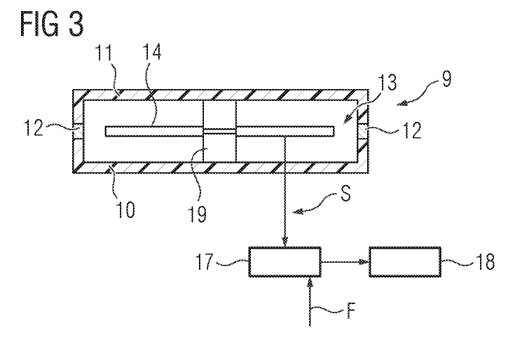

FIG. 3 shows the basic structure of the cladding element 9. According to FIG. 3, the cladding element 9 has an inner shell 10 and an outer shell 11. The inner shell 10 is arranged between the outer shell 11 and the inner structure. The outer shell 11 is therefore visible from the outside when the cladding element 9 is mounted, while the inner shell 10 is not. The inner shell 10 and the outer shell 11 both consist of plastic. The inner shell 10 and the outer shell 11 preferably consist of materials with a recycled content of at least 30%. The recycled content can also be greater than 30% and, in the extreme case, 100% or just below.

The inner shell 10 preferably consists of a material with flameproofing. In this case, it can be necessary to use a relatively low recycled content, for example at most 60% or at most 50%. The outer shell 11, on the other hand, preferably consists of a material without flameproofing. This enables a significantly higher recycled content to be used for the outer shell 11, for example at least 70% or at least 80%.

As can be seen from FIG. 3, the inner shell 10 and the outer shell 11 lie one on top of the other. They are joined together in their edge regions by way of a connection 12. The connection 12 can be for example an adhesively bonded connection. Alternatively, it can be a latching connection. The connection 12 is preferably fluid-tight, where appropriate even gas-tight. To ensure leak-tightness, an additional seal may be required, for example in the manner of an O-ring.

The inner shell 10 and the outer shell 11 form a cavity 13 therebetween. A sensor element 14 is arranged in the cavity 13. However, as can be seen, the sensor element 14 fills the cavity 13 partly but not completely. The sensor element 14 serves to detect when the cladding element 9 collides with the base body 1 or another body, such as for example a patient couch 15 (FIG. 1) or a person, during the movement of the additional part 2 relative to the base body 1. The person can be for example a patient 16 (FIG. 1) lying on the patient couch 15. Another person, for example an operator (not shown), can however also be involved.

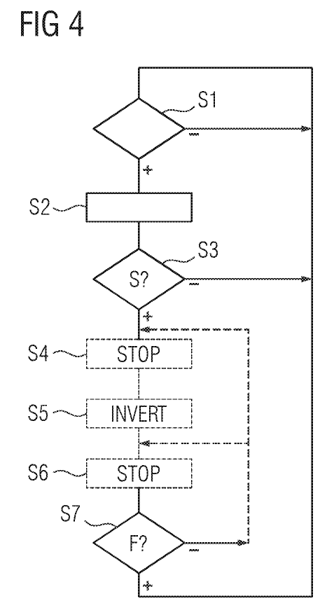

The sensor element 14 issues a sensor signal S in the event of a collision of the cladding element 9. The sensor signal S is received by a control facility 17 (also referred to as a control device or controller) of the device. The mode of operation of the control facility 17 is explained below in connection with FIG. 4 (very schematically).

According to FIG. 4, the control facility 17 checks in a step S1 whether a displacement command is being specified to it, on the basis of which the additional part 2 is to be moved relative to the base body 1. As long as this is not the case, the control facility 17 keeps returning to step S1. Otherwise, in a step S2, the control facility 17 moves the additional part 2 relative to the base body 1 by accordingly actuating a number of drives 18 (FIG. 3).

If the additional part 2 is being moved, the control facility 17 furthermore carries out a step S3. In the step S3, the control facility 17 checks whether the sensor signal S is being transmitted to it by the sensor element 14. If this is not the case, the control facility 17 returns to the step S1. Otherwise, in a step S4, the control facility 17 terminates the movement of the additional part 2 relative to the base body 1. Alternatively, in a step S5, the control facility 17 inverts the movement of the additional part 2 relative to the base body 1 and only then, in a step S6, terminates the movement of the additional part 2 relative to the base body 1. Usually either the step S4 is present or the steps S5 and S6 are present. However, as the steps S4 to 6 are present as alternatives, they are drawn in only as dashed lines in FIG. 4.

The control facility 17 then checks in a step S7 whether an enable signal F is being specified to it, on the basis of which a further movement of the additional part 2 relative to the base body 1 is enabled. As long as this is not the case, the control facility 17 returns to the step S4 or to the step S6. If the enable signal F is specified to it, the control facility 17 returns to the step S1.

The sensor element 14 is fastened in the cavity 13. However, independently of the type of fastening, the sensor element 14 can be removed again from the cavity 13 after the connection 12 is released.

For fastening the sensor element in the cavity 13, as illustrated in FIGS. 3 and 5, the inner shell 10 can for example have positioning elements 19 which interact with corresponding counter-elements 20 of the sensor element 14 such that the sensor element 14 is fastened on account of the interaction of the positioning elements 19 and the counter-elements 20 relative to the inner shell 10. Alternatively or additionally, the outer shell 11 can also have corresponding positioning elements, so that a fastening relative to the outer shell 11 is achieved.

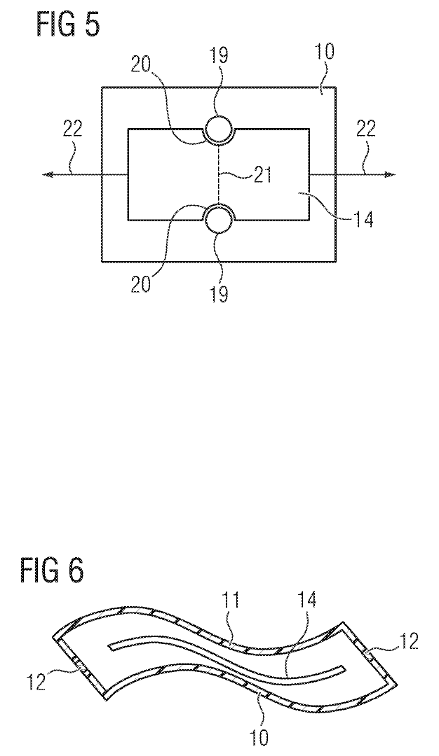

Alternatively or additionally, as illustrated in FIG. 6, it is possible for the inner shell 10 and the outer shell 11 to have an identical three-dimensional curved shape. In this case, the sensor element 14 can be held and fastened in the cavity 13 on account of the molding of the inner shell 10 and the outer shell 11.

FIG. 5 shows a further preferred embodiment, which is preferably realized with the positioning elements 19 and the counter-elements 20. This embodiment can if necessary also be realized in connection with the embodiment of FIG. 6. According to FIG. 5, the sensor element 14 has intended break points 21. The sensor element 14 breaks or tears along the intended break points 21 when a laterally acting tensile force is exerted on the sensor element 14 while the sensor element 14 is disposed between the inner shell 10 and the outer shell 11, as indicated in FIG. 5 by arrows 22.

One or more example embodiments of the present invention have many advantages. It is made easily possible to recycle the cladding element 9. In particular in the case of a latching connection, only the latching connection has to be opened. In the case of an adhesively bonded connection, cutting may be required. A coating may be applied or not as required. Recyclability must be taken into account when considering whether to apply a coating. Furthermore, it is often likewise possible to reuse the sensor element 14 itself. A corresponding material section enables environmental requirements and environmental aspects, in particular sustainability aspects such as for example a low carbon footprint and low toxicity, to be taken into account. In this regard, it is of course necessary to consider that processing characteristics of the materials must meet manufacturing requirements in order to ensure efficient production. Recycling also offers further advantages of both a financial and an ecological nature, so that a business model for recyclers can be established by minimizing the outlay for separation. The provision of prepared separation points offers the possibility to design the material structure in such a way that separability is made easier and thus the purity and consistency of the material characteristics of the components produced is increased.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term "and/or," includes any and all combinations of one or more of the associated listed items. The phrase "at least one of" has the same meaning as "and/or".

Spatially relative terms, such as “beneath,” “below,” “lower,” “under,” “above,” “upper,” and the like, may be used herein for ease of description to describe one element or feature’s relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on," "connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being "directly” on, connected, engaged, interfaced, or coupled to another element, there are no intervening elements present. Other words used to describe the relationship between elements should be interpreted in a like fashion (e.g., "between," versus "directly between," "adjacent," versus "directly adjacent," etc.).

The terminology used herein is for the purpose of describing particular embodiments only and is not intended to be limiting of example embodiments. As used herein, the singular forms "a," "an," and "the," are intended to include the plural forms as well, unless the context clearly indicates otherwise. As used herein, the terms “and/or” and “at least one of” include any and all combinations of one or more of the associated listed items. It will be further understood that the terms "comprises," "comprising," "includes," and/or "including," when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system’s registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility (also referred to as a data processing device or data processor) or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been illustrated and described in detail by way of the preferred exemplary embodiments, the present invention is not restricted by the examples given and other variations can be derived therefrom by a person skilled in the art without departing from the protective scope of the present invention.

What is claimed is:

1. A medical technology device, comprising:
a cladding element including a sensor element, the sensor element configured to
detect a collision of the cladding element during at least one of movement of the medical technology device or movement of an additional part, and
issue a sensor signal in response to the collision of the cladding element, wherein
the cladding element has an inner shell and an outer shell,
the inner shell and the outer shell lie one on top of another and are joined together at edge regions by way of a connection,
the inner shell and the outer shell form a cavity therebetween,
the sensor element is arranged in the cavity to at least partially fill the cavity,
the sensor element is fastened in a releasable manner in the cavity, and
after the connection is released, at least one of the inner shell, the outer shell, the connection or the sensor element are at least one of reusable or recyclable.

2. A medical technology device, comprising:
a base body; and
at least one additional part configured to move relative to the base body, the at least one additional part having a cladding element including a sensor element, wherein
the sensor element is configured to detect when the cladding element collides with the base body or another body during movement of the at least one additional part relative to the base body.

3. The medical technology device as claimed in claim 1, wherein
after the connection is released, at least one of the inner shell, the outer shell or the sensor element are configured to be at least one of separated or divided in a non-destructive manner.

4. The medical technology device as claimed in claim 1, wherein
by releasing the connection, separate individual components are obtained from the cladding element, and
the separate individual components have at least one of greater purity or recyclability than an unreleased cladding element.

5. The medical technology device as claimed in claim 1, wherein
the inner shell and the outer shell include materials with a recycled content of at least 30%.

6. The medical technology device as claimed in claim 1, wherein
the inner shell and the outer shell are based on at least one plastic material.

7. The medical technology device as claimed in claim 1, wherein
the inner shell and the outer shell have an identical three-dimensional curved shape, and
the sensor element is held and fastened in the cavity on account of a molding of the inner shell and the outer shell.

8. The medical technology device as claimed in claim 1, wherein
at least one of the inner shell or the outer shell have positioning elements that interact with corresponding counter-elements of the sensor element such that the sensor element is fastened on account of an interaction of the positioning elements and the corresponding counter-elements relative to the at least one of the inner shell or the outer shell.

9. The medical technology device as claimed in claim 1, wherein
the sensor element has intended break points, along which the sensor element is configured to break or tear when a laterally acting tensile force is exerted on the sensor element while the sensor element is disposed between the inner shell and the outer shell.

10. The medical technology device as claimed in claim 1, wherein
the connection of the inner shell and the outer shell is an adhesively bonded connection or a latching connection.

11. The medical technology device as claimed in claim 1, wherein
the inner shell includes a material with flameproofing, and
the outer shell includes a material without flameproofing.

12. The medical technology device as claimed in claim 1, further comprising:
a control device configured to
control the movement of the additional part relative to a base body through corresponding actuation of a number of drives,
receive the sensor signal, and
terminate or invert the movement of the additional part relative to the base body in response to the sensor signal.

13. The medical technology device as claimed in claim 1, wherein
at least one of the inner shell or the outer shell are reusable without shredding after dismantling of the cladding element.

14. The medical technology device as claimed in claim 1, wherein the cladding element has a modular structure.

15. The medical technology device as claimed in claim 3, wherein
by releasing the connection, separate individual components are obtained from the cladding element, and
the separate individual components have at least one of greater purity or recyclability than an unreleased cladding element.

16. The medical technology device as claimed in claim 3, wherein
the inner shell and the outer shell include materials with a recycled content of at least 30%.

17. The medical technology device as claimed in claim 3, wherein
the inner shell and the outer shell are based on at least one plastic material.

18. The medical technology device as claimed in claim 3, wherein
the inner shell and the outer shell have an identical three-dimensional curved shape, and
the sensor element is held and fastened in the cavity on account of a molding of the inner shell and the outer shell.

19. The medical technology device as claimed in claim 3, wherein
at least one of the inner shell or the outer shell have positioning elements that interact with corresponding counter-elements of the sensor element such that the sensor element is fastened on account of the interaction of the positioning elements and the corresponding counter-elements relative to the at least one of the inner shell or the outer shell.

20. The medical technology device as claimed in claim 3, further comprising:
a control device configured to
control the movement of the additional part relative to a base body through corresponding actuation of a number of drives,
receive the sensor signal, and
terminate or invert the movement of the additional part relative to the base body in response to the sensor signal.
